(19) United States
(12) Patent Application Publication
(10) Pub. No.: US 2008/0152655 A1
(43) Pub. Date: Jun. 26, 2008

(54) Combination Therapy With Anti-Ctla4 and Anti-4-1BB Antibodies

(75) Inventors: Yang Liu, Ann Arbor, MI (US); Pan Zheng, Ann Arbor, MI (US); Ergun Kocak, Columbus, OH (US)

Correspondence Address: CALFEE HALTER & GRISWOLD, LLP, 800 SUPERIOR AVENUE, SUITE 1400, CLEVELAND, OH, 44114, US

(73) Assignee: The Ohio State University Research Foundation, Columbus, OH (US)

(21) Appl. No.: 10/536,115

(22) PCT Filed: Sep. 7, 2005
(86) PCT No.: PCT/US2005/031899

§ 371 Date: Dec. 31, 2007

Related U.S. Application Data
(60) Provisional application No. 60/608,000, filed on Sep. 8, 2004.

(51) Int. Cl. A61K 39/395; A61P 35/00

(52) U.S. Cl. 424/158.1

(57) ABSTRACT

Methods for screening monoclonal antibodies to CTLA4, monoclonal antibodies to human CTLA4, therapeutic compositions containing the same.

This application claims priority to U.S. Provisional Application No. 60/608,000, filed Sep. 8, 2004, the entire disclosure of which is incorporated herein by reference.

Work leading to this invention was supported, at least in part, by grants from the National Cancer Institute: R01CA69091, R01CA58033, and R41CA93107. The government has certain rights in this invention.

The present invention relates to methods of treating cancer. The methods generally comprise administering to a patient in need of treatment anti-4-1BB antibody and anti-CTLA4 antibody in amounts effective to produce an anti-cancer effect. Additionally, in some embodiments, the administration of anti-4-1BB antibody and anti-CTLA4 antibody result in a lower level of autoimmunity as compared to administration of anti-CTLA4 or anti-4-1BB antibody alone.

Some embodiments of the invention relate to methods for reducing an autoimmune side effect in administration of anti-CTLA4 antibodies comprising administering an effective amount of anti-4-1BB antibody to a patient anticipating or experiencing anti-CTLA4 autoimmune side effects. Some embodiments of the invention relate to methods for reducing an autoimmune side effect in administration of anti-4-1BB antibodies comprising administering an effective amount of anti-CTLA4 antibody to a patient anticipating or experiencing anti-4-1BB autoimmune side effects. Still further, some embodiments of the invention relate to methods for enhancing cancer immunity in a patient while reducing autoimmunity in said patient comprising administering anti-4-1BB antibody and anti-CTLA4 antibody in effective amounts to a patient in need of treatment.

As used herein, the term “anti-cancer” effect includes, but is not limited to, preventing or reducing metastasis, decreasing cancer burden, decreasing cancer growth, and reduction in new cancer formation. Cancer includes all types of cancer and is not limited to solid tumors. As used herein, the term “autoimmunity” is used as it is normally used in the art, and can be measured by testing for anti-DNA antibodies, using known methods. Autoimmunity can also be measured by observing inflammation in noncancerous tissues.

According to the invention, the method of administration of the anti-4-1BB antibody and anti-CTLA4 antibody is not critical. In some embodiments, the antibodies are administered simultaneously and in other embodiments, the antibodies are administered at different times. When administered simultaneously, the antibodies can be in the same composition or in separate compositions. When administered at different times, the time between administrations can range from seconds to minutes to hours to days.

The invention is also directed to composition for treating cancer comprising effective amounts of anti-4-1BB antibody and anti-CTLA4 antibody, and at least one pharmaceutically acceptable excipient, wherein the amount of the antibodies in the composition is sufficient to produce a lower level of autoimmunity as compared to a composition of either anti-CTLA4 or anti-4-1BB antibody alone.

It should be noted that the combination administration yielded surprising results for at least two reasons. First, the therapeutic effect of the combination appears to produce synergistic results—a therapeutic effect in the combination that is greater than the expected additive effect of the individual antibodies. Second, both ant-CTLA4 and anti-4-1BB appear to produce an autoimmune effect; yet when delivered in combination, the autoimmune effect is less than either one alone.

The compositions of the invention can be administered, and the methods of the invention practiced, orally, parenterally (IV, IM, depot-IM, SQ, and depot-SQ), sublingually, intranasally (inhalation), intrathecally, topically, or rectally. Dosage forms known to those of skill in the art are suitable for delivery of the inventive compounds employed in the methods of the invention.

Compositions are provided that contain therapeutically effective amounts of the inventive compositions. The compositions can be formulated into suitable pharmaceutical preparations such as tablets, capsules, or elixirs for oral administration or in sterile solutions or suspensions for parenteral administration. The compounds described herein can be formulated into pharmaceutical compositions using techniques and procedures well known in the art.

To prepare compositions, one or more inventive compounds employed in the methods of the invention are mixed with a suitable pharmaceutically acceptable carrier. Upon mixing or addition of the compound(s), the resulting mixture may be a solution, suspension, emulsion, or the like. Liposomal suspensions may also be used as pharmaceutically acceptable carriers. These may be prepared according to methods known to those skilled in the art. The form of the resulting mixture depends upon a number of factors, including the intended mode of administration and the solubility of the compound in the selected carrier or vehicle. The effective concentration is sufficient for lessening or ameliorating at least one symptom of the disease, disorder, or condition treated and may be empirically determined.

Pharmaceutical carriers or vehicles suitable for administration of the compositions provided herein include any such carriers suitable for the particular mode of administration. In addition, the active materials can also be mixed with other active materials that do not impair the desired action, or with materials that supplement the desired action, or have another action. The compounds may be formulated as the sole pharmaceutically active ingredient in the composition or may be combined with other active ingredients.

The inventive compositions employed in the methods of the invention may be prepared with carriers that protect them against rapid elimination from the body, such as time-release formulations or coatings. Such carriers include controlled release formulations, such as, but not limited to, microencapsulated delivery systems. The antibodies can be included in the pharmaceutically acceptable carder in an amount sufficient to exert a therapeutically useful effect in the absence of undesirable side effects on the patient treated. The therapeutically effective concentration may be determined empirically by testing the compounds in known in vitro and in vivo model systems for the treated disorder.

The compositions of the invention can be enclosed in multiple or single dose containers. The enclosed compositions can be provided in kits, for example, including component parts that can be assembled for use. For example, an inventive composition in lyophilized form and a suitable diluent may be provided as separated components for combination prior to use. A kit may include an inventive composition and another therapeutic agent for co-administration. The inventive composition and additional therapeutic agent may be provided as separate component parts. A kit may include a plurality of containers, each container holding one or more unit dose of the inventive compositions employed in the invention. The containers can be adapted for the desired mode of administration, including, but not limited to tablets, gel capsules, sustained-release capsules, and the like for oral administration; depot products, pre-filled syringes, ampoules, vials, and the like for parenteral administration; and patches, medipads, creams, and the like for topical administration.

The concentration of the antibodies in the composition will depend on absorption, inactivation, and excretion rates of the antibodies, the dosage schedule, and amount administered as well as other factors known to those of skill in the art.

The compositions may be administered at once, or may be divided into a number of smaller doses to be administered at intervals of time. It is understood that the precise dosage and duration of treatment is a function of the disease being-treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

The antibodies can also be mixed with other active materials that do not impair the desired action, or with materials that supplement the desired action. The inventive compositions can be used, for example, in combination with another antitumor agent, a hormone, a steroid, or a retinoid. The antitumor agent may be one of numerous chemotherapy agents such as an alkylating agent, an antimetabolite, a hormonal agent, an antibiotic, colchicine, a vinca alkaloid, L-asparaginase, procarbazine, hydroxyurea, mitotane, nitrosoureas or an imidazole carboxamide.

Solutions or suspensions used for parenteral, intradermal, subcutaneous, or topical application can include any of the following components: a sterile diluent such as water for injection, saline solution, fixed oil, a naturally occurring vegetable oil such as sesame oil, coconut oil, peanut oil, cottonseed oil, and the like, or a synthetic fatty vehicle such as ethyl oleate, and the like, polyethylene glycol, glycerin, propylene glycol, or other synthetic solvent; antimicrobial agents such as benzyl alcohol and methyl parabens; antioxidants such as ascorbic acid and sodium bisulfite; chelating agents such as ethylenediaminetetraacetic acid (EDTA); buffers such as acetates, citrates, and phosphates; and agents for the adjustment of tonicity such as sodium chloride and dextrose. Parenteral preparations can be enclosed in ampoules, disposable syringes, or multiple dose vials made of glass, plastic, or other suitable material. Buffers, preservatives, antioxidants, and the like can be incorporated as required.

Where administered intravenously, suitable carriers include, but are not limited to, physiological saline, phosphate buffered saline (PBS), and solutions containing thickening and solubilizing agents such as glucose, polyethylene glycol, polypropyleneglycol, and mixtures thereof. Liposomal suspensions including tissue-targeted liposomes may also be suitable as pharmaceutically acceptable carriers. These may be prepared according to methods known in the art.

The inventive compositions may be prepared with carriers that protect the compound against rapid elimination from the body, such as time-release formulations or coatings. Such carriers include controlled release formulations, such as, but not limited to, implants and microencapsulated delivery systems, and biodegradable, biocompatible polymers such as collagen, ethylene vinyl acetate, polyanhydrides, polyglycolic acid, polyorthoesters, polylactic acid, and the like. Methods for preparation of such formulations are known to those skilled in the art.

The inventive compositions and methods can be used to inhibit neoplastic cell proliferation in an animal. The methods generally comprise administering to an animal having at least one neoplastic cell present in its body. The animal can be a mammal, including a domesticated mammal. The animal can be a human.

The term “neoplastic cell” is used to denote a cell that shows aberrant cell growth. The aberrant cell growth of a neoplastic cell includes increased cell growth. A neoplastic cell may be, for example, a hyperplastic cell, a cell that shows a lack of contact inhibition of growth in vitro, a benign tumor cell that is incapable of metastasis in vivo, or a cancer cell that is capable of metastases in vivo and that may recur after attempted removal. The term “tumorigenesis” is used to denote the induction of cell proliferation that leads to the development of a neoplastic growth.

The terms “therapeutically effective amount” and “therapeutically effective period of time” are used to denote treatments at dosages and for periods of time effective to reduce neoplastic cell growth. The present invention provides compositions and methods for treating a cell proliferative disease or condition in an animal. The term “cell proliferative disease or condition” is meant to refer to any condition characterized by aberrant cell growth, preferably abnormally increased cellular proliferation. Examples of such cell proliferative diseases or conditions include, but are not limited to, cancer, restenosis, and psoriasis. In some embodiments, the invention provides a method for inhibiting neoplastic cell proliferation in an animal comprising administering to an animal having at least one neoplastic cell present in its body a therapeutically effective amount of a compound of the invention. Cancers treatable according to the invention include, but are not limited to, prostate cancer, lung cancer, acute leukemia, multiple myeloma, bladder carcinoma, renal carcinoma, breast carcinoma, colorectal carcinoma, neuroblastoma, brain cancer, ovarian cancer, or melanoma.

It should be apparent to one skilled in the art that the exact dosage and frequency of administration will depend on the particular compositions employed in the methods of the invention administered, the particular condition being treated, the severity of the condition being treated, the age, weight, general physical condition of the particular patient, and other medication the individual may be taking as is well known to administering physicians who are skilled in this art.

It is to be understood that both the foregoing general description and the following detailed description are exemplary and explanatory only and are not restrictive of the invention, as claimed.

The accompanying drawings, which are incorporated in and constitute a part of this specification, illustrate one (several) embodiment(s) of the invention and together with the description, serve to explain the principles of the invention.

### BRIEF DESCRIPTION OF THE DRAWINGS

FIG. 1: Synergistic therapeutic effect of anti-4-1BB and anti-CTLA-4 antibodies in both minimal disease (a) and established tumor (b) models. a. Therapy of minimal diseases. C57BL/6 mice were inoculated with 5×10⁵ MC38 cells. On days 2, 9 and 16 after tumor cell injection, control hamster and rat IgG, anti-CTLA-4, anti-4-1BB antibodies were injected. Tumor sizes were measured by physical examination. Data shown were growth kinetics of tumors, the sizes presented are products of long and short diameters of the tumor. b. Therapy of established tumors. As in a, except that therapy started on day 14 after inoculation of tumor cells when tumor reached sizes of between 9-60 mm² in sizes. The synergistic effect of the two antibodies on established tumors have been repeated 3 times.

FIG. 2: CD8 T cells, but not CD4 or NK cells, are essential for antibody-induced tumor rejection. Tumor-bearing mice were depleted of either CD4, CD8, or NK cells by three injections of antibodies specific for either CD4, CD8 or NK1.1 on days 9, 12, and 16 after tumor cell inoculation. Therapeutic antibodies were inoculated on days 9, 16 and 23. Data shown are means and S.D. of tumor sizes (n=3).

FIG. 3: Combination therapy reduces production of anti-DNA antibodies and lupus-like pathology in the kidney. a. Serum anti-DNA antibodies in tumor-bearing mice receiving therapy of control IgG, anti-CTLA-4, anti-4-1BB, or anti-4-1BB+anti-CTLA-4 antibodies. Data shown were means and S.D. of O.D.492 from groups of 4 mice and are representative of three independent experiments. b. Deposition of immune complex in the glomeruli as revealed by deposition of C3 (top panels) and IgG (middle panels). Merged images are provided in the lower panels.

FIG. 4: Combination therapy reduces inflammation in the liver and the lung associated with treatment with either anti-4-1BB or anti-CTLA-4 antibodies. a. H&E staining of lung (upper panels) of liver sections of tumor bearing mice that received control IgG, anti-CTLA-4, anti-4-1BB, or anti-CTLA-4+anti-4-1BB antibodies. b. Summary of pathology scores. Data shown are pathological scores of liver and lung, where most inflammation is seen, according to the following criteria. Lung: 0, no inflammation; 1, mild inflammation with perivascular lymphocytic infiltration, <10% lung sections involved; 2, mild to intermediate inflammation with increased infiltration of lymphocytes, plasma cells and interstitial fibrosis and mild consolidation of lung parenchyma. 10-25% lung sections involved; 3 intermediate to severe inflammation with increased infiltration of lymphocytes, plasma cells and some neutrophils and eosinophils. Interstitial fibrosis with 30-60% lung sections involved; 4, severe acute inflammation with predominant infiltration of neutrophils, pulmonary edema, consolidation of lung parenchyma. More than 60% lung sections involved. Liver: 0, no inflammation; 1, mild inflammation with less than 15 small foci of 5-10 lymphocytes around triad, central vein or in parenchyma; 2, mild to intermediate inflammation with less than five medium size foci of 10-30 lymphocytes around triad, central vein, or parenchyma, or mild fibrosis is present in medium size inflammatory foci, or more than 15 small foci of inflammation; 3, intermediate to severe inflammation with large foci of 30-70 cells consisting of lymphocytes, neutrophils and eosinophils; 4, micro-abscess formation with more than 100 cells consist of predominantly neutrophils and eosinophils.

FIG. 5: Combination of anti-4-1BB and anti-CTLA-4 antibodies enhanced function of Treg in the mice. a, b. Expression of 4-1BB and CTLA-4 on the cell surface of Treg isolated from untreated C57BL/6 mice. Spleen cells were analyzed for expression of CD4, CD25, 4-1BB and CTLA-4 as detailed in experimental procedures. A profile for expression of 4-1BB and CTLA-4 on gated Treg were shown in b, while that of isotype control is shown in a. c and d, % (c) and biological activity (d) of Treg in mice treated with either control Ig or C57BL/6 mice were treated with 3 consecutive injections of anti-CTLA-4+anti-4-1BB antibodies. One week after the last injection the spleen were harvested and analyzed for the % of CD4⁺CD25⁺ T cells (c). The Treg were isolated from spleen by MACS beads and tested for their ability to inhibit proliferation of CD4⁺CD25⁻ spleen cells, as detailed in materials and methods. Data shown are means and SEM of triplicate cultures.

FIG. 6: Combination therapy with anti-4-1BB and anti-human CTLA4 antibody in human CTLA4. Human CTLA4 knockin mice were inoculated with 5×105 MC38 tumor cells subcutaneously. Two days later, groups of 7 mice were treated with either control rat+mouse IgG, 2A(anti-4-1BB)+mouse IgG, L3D10 (anti-human CTLA4)+rat IgG, and L3D10+2A, as indicated in the arrows. Data shown are mean tumor volume and SEM. Statistical analysis revealed significant difference in the following comparison, 2A vs control IgG, L3D10 vs control IgG, 2A+L3D10 vs all three groups, P<0.001.

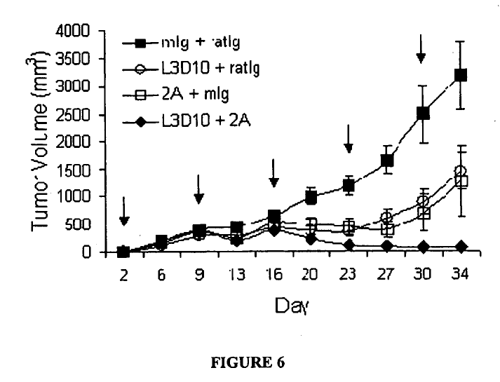

FIG. 7: Tumor-free mice in the double antibody-treated group developed long lasting immunity to MC38 tumors. At 110 days after the first tumor cell challenge, the double antibody-treated, tumor-free mice or control naive mice were challenged with 5×10⁵ tumor cells subcutaneously. Tumor growth were monitored by physical examination. Note that all of the mice that rejected the tumors in the first round are completely resistant to re-challenge, while all naïve mice had progressive tumor growth.

FIG. 8: Combination therapy reduces host responses to anti-CTLA4 antibodies. Hamster-anti-mouse-CTLA-4 (a) or rat-anti-mouse-4-1BB (b) antibodies were coated in ELISA plates. Different dilutions of sera from groups of 5 mice each, as those used in FIG. 8a, were added to the plates. The relative amounts of antibody bound were determined using a secondary step reagent (biotinylated goat anti-mouse antibodies that were depleted of reactivity to rat and hamster IgG by adsorption). Data shown are means and SEM of optical density at 490 nm. Similar reduction of host antibody response to anti-CTLA-4 and 4-1BB was observed when tumor-free mice were treated with the same antibodies (data not shown).

### DESCRIPTION OF THE EMBODIMENTS

Reference will now be made in detail to the present embodiment(s) (exemplary embodiments) of the invention, example(s) of which is (are) illustrated in the accompanying drawings.

### EXAMPLES

Materials and Methods

Antibodies. Anti-4-1BB mAb-producing hybridoma, 2A³⁰, was provided by Dr. Lieping Chen. Anti-CTLA-4 mAb-producing hybridoma, 4F10³¹, was a gift from Dr. Jeff Bluestone. Both anti-4-1BB and anti-CTLA-4 mAbs were purified from supernatant by a Protein G column. Hamster and Rat IgG were purchased from Rockland Immunochemicals, Inc. (Gilbertsville, Pa.). Hybridomas that secrete depleting antibodies specific for NK1.1 (PK136), CD25 (PC61), CD4 (GK1.5) and CD8 (2.4.3) were purchased from American Tissue Culture Collection (ATCC, Manassas, Va.). The anti-4-1BB antibody 2A was biotinylated according to an established procedure. Fluorochrome conjugated anti-CTLA-4, CD4, CD25, were purchased from BD-Pharmingen (La Jolla, Calif.).

Experimental Animals. 6-8-week-old female C57BL/6 mice were purchased from the National Cancer Institute (Frederick, Md.). Animals were maintained under pathogen-free conditions in accordance with federal guidelines.

Cell Lines and Tumorigenicity Assay. C57BL/6 colon cancer MC38 cells were purchased from ATCC. MC38 cells (5×10⁵) suspended in serum free RPMI (100 μl) were injected subcutaneously in the flanks of mice. Starting either day 2 (minimal diseases model) or 14 (large established tumor model), the tumor-bearing mice received 3 weekly injections of either hamster (800 μg/mouse/injection) plus rat (200 μg/mouse/injection) IgG, anti-CTLA-4 mAb 4F10 (800 μg/mouse/injection), anti-4-1BB antibody 2A (200 μg/mouse/injection), or both antibodies. Tumor size and incidence were determined every 2-5 days by physical examination.

Flow Cytometry. Lymphocyte subsets were analyzed by flow cytometry using a FACSCalibur. With exception of anti-CTLA-4 antibody, all antibodies were incubated with the spleen cells at 4° C. for 30-60 min. The unbound antibodies were washed away with PBS containing 1% fetal calf serum and 0.01 % of sodium azide. To analyze expression of CTLA-4 on the surface of Treg, the spleen cells were incubated with 1 μg/ml of PE-conjugated 4F10 or isotype control in the presence of 0.1 μg/ml of anti-CD3 and 1000 fold excess of hamster IgG to block nonspecific binding. After washing away the unbound antibodies, the cells were placed at 4° C. for staining with biotinylated anti-4-1BB antibodies and APC-conjugated Streptavidin. The Treg were marked by anti-CD4-FITC and anti-CD25-cychrome.

Depleting of Lymphocyte Subset in Vivo. In vivo depletion was achieved by injection of anti-CD4 (0.5 mg/injection/mouse), anti-CDB (0.5 mg/mouse/injection), and anti-NK1.1 (0.1 mglinjection/mouse) on days 9, 12, and 16 after tumor cell inoculation.

Detection of Anti-Double Stranded DNA Antibodies. Anti-DNA antibodies were measured by ELISA according to published procedures²⁰.

Immunofluorescence for Antibody and Complement Deposition in the Kidney Glomerulus. Frozen sections of kidney were prepared at 4 weeks after tumor cell inoculation and fixed in acetone. After blocking with 10% normal goat serum, the sections were stained with Rhodamine-conjugated goat anti-mouse IgG or FITC-conjugated goat anti-mouse C3 antibodies (ICN Biomedicals, Inc., Aurora, Ohio).

Histology. Internal organs from tumor-bearing mice receiving different antibodies were fixed with 10% formalin. The fixed tissues were sectioned and stained by H&E. The pathology score was based on the size and number of inflammatory foci as detailed in Figure legends.

Results

Synergistic Effect of Anti-CTLA-4 and Anti-4-1BB Antibodies in Induction of CD8+ T Cell-Mediated Tumor Rejection.

A model of minimal diseases and that of large established tumors was used to test the anti-tumor effect of combining anti-4-1BB and anti-CTLA-4 mAb treatments. C57BL/6 mice were challenged with a subcutaneous inoculation of MC38 colon cancer cells, and at different times after tumor cell inoculation, antibodies were injected into tumor-bearing mice and the tumor size and incidence were monitored by physical examination.

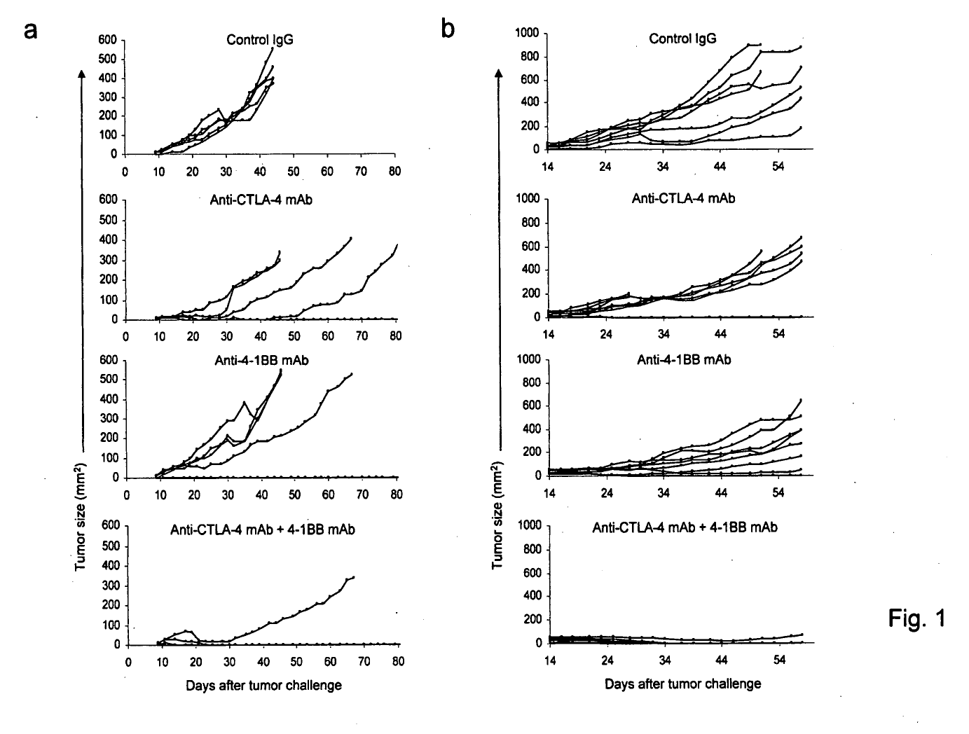

In the minimal disease model, the mice were treated with control IgG, anti-4-1BB mAb alone, anti-CTLA-4 mAb alone, or anti-4-1BB combined with anti-CTLA-4 mAbs starting at 48 hours after inoculation of tumor cells. The antibodies were administered i.p. on days 2, 9, and 16. As shown in FIG. 1a, treatment with either anti-4-1BB or anti-CTLA-4 mAb alone resulted in a slight delay in tumor growth with one mouse in each group rejecting tumors. Interestingly, 4 out of 5 mice treated with both anti-CTLA-4 and anti-4-1BB mAbs were tumor-free at the conclusion of the experiment. Thus, in the setting of minimal tumor-burden, the combination of anti-4-1BB and anti-CTLA-4 mAbs results in tumor rejection in most cases and delays tumor growth and prolongs survival of those mice that did not completely reject the tumor.

To determine if the anti-tumor effects of combination mAb treatment against small tumor burden could be extended to therapeutic applications against larger tumor burdens, experiments treating mice with established tumors were performed. Wild type C57BL/6 mice were challenged with a subcutaneous inoculation of MC38 colon cancer cells. Tumors were allowed to grow for 14 days, at which point, mice with established tumors (usually >7 mm in diameter) were selected and divided randomly into four treatment groups: control IgG, anti-4-1BB mAb only, anti-CTLA-4 mAb only, and anti-4-1BB mAb combined with anti-CTLA-4 mAb. The antibodies were administered i.p. on days 14, 21, and 28 after tumor challenge. As shown in FIG. 1b, treatment with anti-CTLA-4 mAb did not impede tumor growth when compared to control IgG treatment, although rejection was seen in one of the eight mice in the group. Treatment with anti-4-1BB mAb slowed tumor growth somewhat, but only one in eight mice rejected the tumor. In contrast, combination therapy with both anti-CTLA-4 and anti-4-1BB mAbs lead to the eradication of tumors in 8/9 mice and prevention of further tumor growth in the remaining mouse. These results indicate that anti-CTLA-4 and anti-4-1BB mAbs act synergistically in causing the rejection of large established tumors.

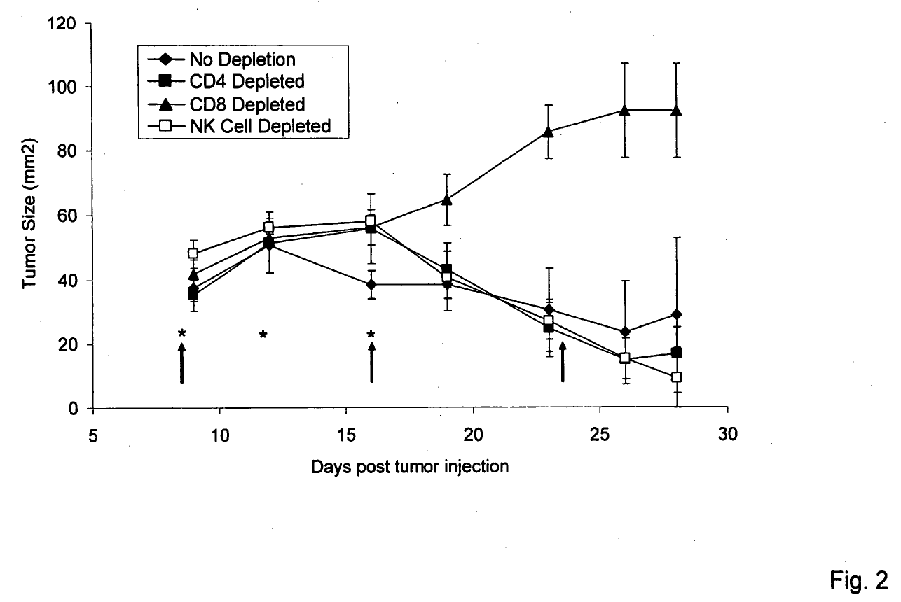

To determine which subset of immune cells was contributing to the anti-tumor effect elicited by combination mAb treatment, the major subsets of lymphocytes were deleted with monoclonal antibodies. Similar to the above experiments, MC38 tumor cells were injected subcutaneously. Once tumors were palpable, tumor-bearing mice were separated into four groups. Each group had a series of intraperitoneal antibody injections to deplete differing subsets of immune cells, including no depletion with polyclonal rat IgG, CD4+ T cell depletion with anti-CD4 mAb (GK 1.5), CD8+ T cell depletion with anti-CD8 mAb (2.4.3), and NK cell depletion with anti-NK1.1 mAb (PK136). In addition, all mice in all groups were treated with anti-CTLA-4 plus anti-4-1BB mAbs once weekly for three weeks. Adequate depletion of immune cell subsets was evaluated by flow cytometry of peripheral blood taken from mice immediately prior to completion of the experiment (data not shown). As expected, mice with no depletion of immune cells responded to treatment with anti-CTLA-4 combined with anti-4-1BB mAb (FIG. 2). Similarly, depletion of NK cells and CD4+ T cells did not affect the anti-tumor activity of combination anti-CTLA-4 plus anti-4-1BB mAb therapy. The depletion of CD8⁺ T cells, however, abrogated the anti-tumor activity of combination antibody therapy. These data demonstrate that the tumor-eradicating effects of anti-CTLA-4 and anti-4-1BB mAb treatment is CD8⁺ T cell-dependent.

Combination Therapy Uncouples Autoimmunity and Cancer Immunity.

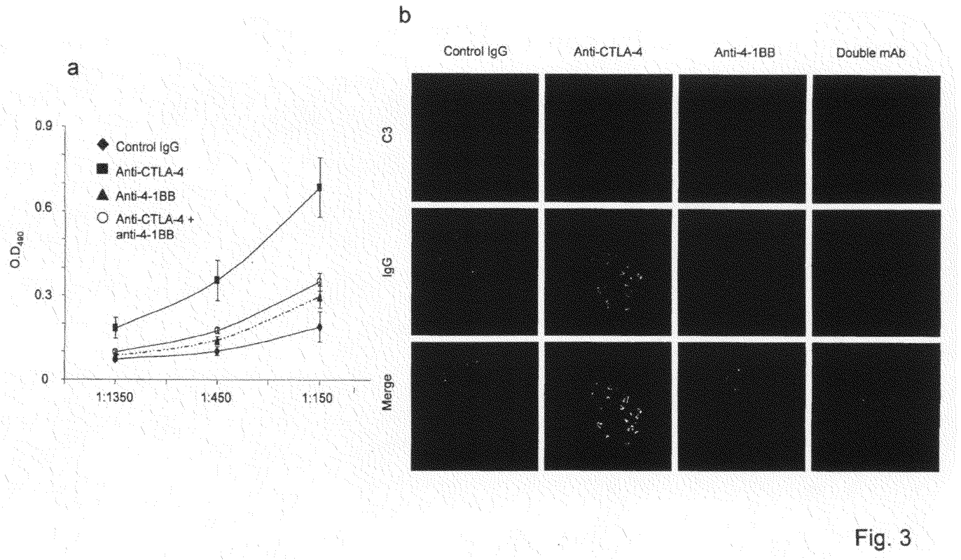

Two approaches were taken to determine whether increased cancer immunity is associated with increased autoimmunity. First, sera samples were obtained at three weeks after the last antibody injection and anti-double-stranded DNA antibodies and renal antibody deposition were measured. As shown in FIG. 3a, anti-CTLA-4 antibody significantly increased serum levels of anti-DNA antibodies. While low levels of anti-DNA antibodies were detected in anti-4-1BB-treated mice, they were similar to those observed in the control IgG-treated group. Significantly, anti-4-1BB inhibits production of anti-DNA antibodies induced by anti-CTLA-4 mAb. To confirm the pathological significance of the anti-DNA antibodies, the antibody and complement deposition in the kidneys of antibody-treated mice, harvested at just over 8 weeks after completion of antibody treatment, were investigated. As shown in FIG. 3b, immune complex depositions were observed in the group that received anti-CTLA-4 antibody alone. This was significantly reduced in the group that received either anti-4-1BB alone, or anti-4-1BB+anti-CTLA4. Thus, in agreement with the mouse model of lupus^{20,21}, anti-4-1BB antibodies inhibited the anti-DNA antibody production.

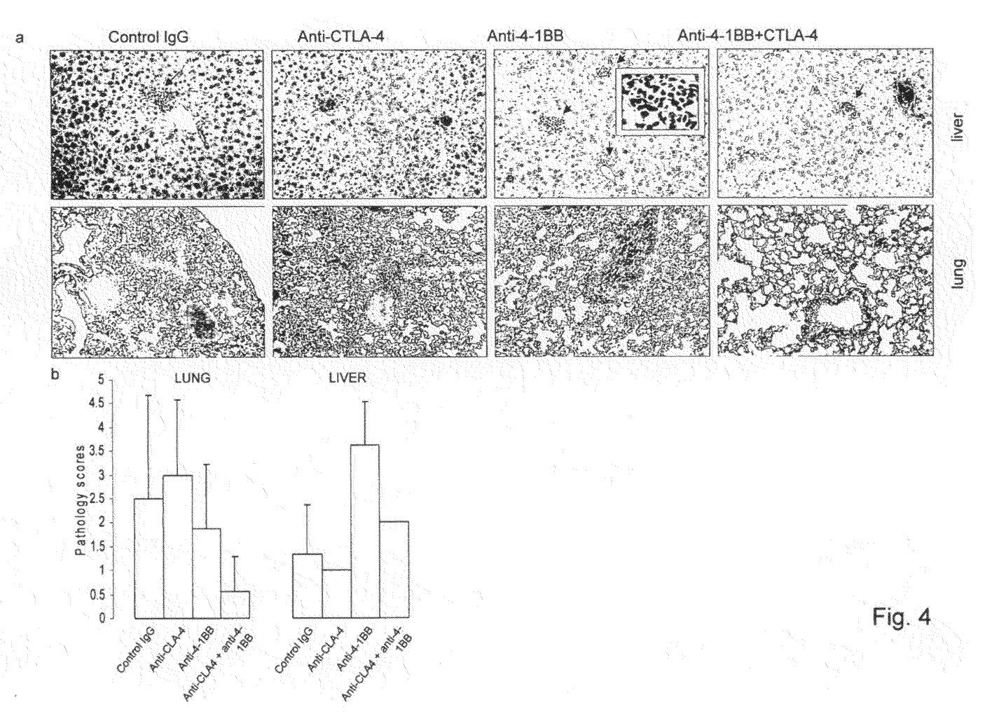

In the experiments with established tumors, multiple organs from the mice that received control IgG, anti-CTLA-4, anti-4-1BB or both, were analyzed for inflammation (FIG. 4). Although small foci of inflammation could be seen in the intestine and the stomach (data not shown), most inflammation was seen in the lung and the liver. Inflammation in the lung was observed in tumor-bearing mice that received control IgG, and treatment with anti-CTLA-4, but not-anti-4-1BB, exacerbated inflammation in the lung. The lung inflammation, however, was eliminated by a combination of anti-CTLA-4 and anti-4-1BB antibody (P<0.05 compared with CTLA-4 antibody alone). Surprisingly, anti-4-1BB antibody, but not anti-CTLA-4 antibody, greatly enhanced inflammation in the liver as judged both by the number and size of foci. This was abrogated by co-injected anti-CTLA-4 antibody (P<0.001). Thus, combination therapy with both anti-4-1BB and CTLA-4 can enhance anti-tumor immunity while reducing inflammation to normal host organs.

Combination of Anti-4-1BB and anti-CTLA-4 Increases Activity of Regulatory T Cells.

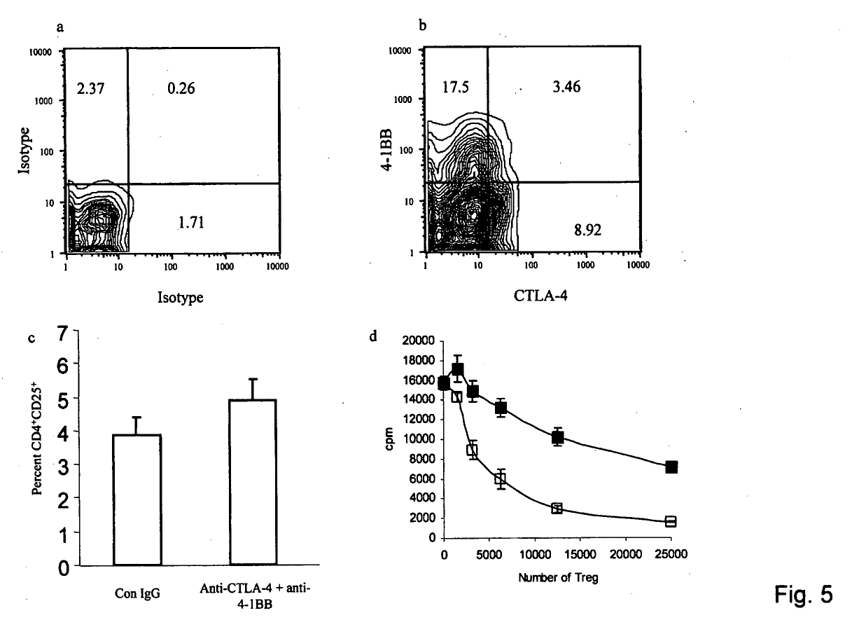

Both CTLA4 and 4-1BB are over-expressed in Treg²²⁻²⁴. Flow cytometry was used to determine the distribution of the 4-1BB and CTLA-4molecules on Trng. Since CTLA-4 normally reside intracellularly²⁵, spleen cells were stimulated with anti-CD3 at 37° C. in the presence of labeled anti-CTLA-4 antibodies. Excess levels of normal hamster IgG and anti-FcR mAb were added to prevent non-specific binding. After unbound anti-CTLA-4 antibodies were washed away, biotinylated anti-4-1BB antibodies were added at 4° C. As shown in FIG. 5a, 4-1BB and CTLA-4 were both expressed on the surface of Treg after short-term stimulation, although their expression appeared to be independent of each other. While expression of CTLA-4 on the cell surface required stimulation, expression of 4-1BB was constitutive on Treg (data not shown), as others have reported²⁶.

Expression of 4-1BB and CTLA-4 on Treg raised the intriguing possibility that suppression of autoimmunity by the two antibodies can be achieved by modulating the activity of Treg. To test this possibility, normal mice were treated with either control IgG or the two mAbs. After three injections, the spleen cells were harvested to analyze the number and activity of Treg. As shown in FIG. 5c, the number of Treg was slightly increased in antibody-treated group. Interestingly, anti-4-1BB and CTLA-4 antibodies drastically increased the Treg activity. On a cell-to-cell basis, Treg from the double antibody-treated group were 4-8-fold more efficient than those isolated from control Ig-treated group. These results demonstrate that combination therapy increases Treg activity.

Discussion

These results demonstrate that combination therapy with anti-4-1BB and anti-CTLA-4 antibodies enhanced cancer immunity but reduced autoimmunity. The results indicate that cancer immunity and autoimmunity are not necessarily linked even though the majority of tumor antigens can be found to be expressed at limited levels on normal tissues. Several lines of recent data, primarily in autoimmune depigmentation associated with melanoma antigen, are consistent with this notion. Thus, although antibodies against TYRP-1/gp75 can produce both tumor rejection and autoimmune depigmentation, the autoimmune destruction requires 5-fold more antibodies and can be distinguished from tumor rejection by the requirement for FcR and complement^{9,27}. Likewise, after immunization with antigen TYRP-2/DCT, T cell-mediated tumor rejection can be perforin-independent, while autoimmune depigmentation requires perforin^{28,29}. While these studies raised a theoretical possibility to unravel cancer immunity and autoimmunity, the present invention provides a novel and generally applicable approach to enhance cancer immunity in the absence of autoimmunity.

An interesting issue is the immunological basis by which combination therapy uncouples cancer immunity and tumor immunity. The reduction of anti-DNA antibody and kidney deposition of immune complex by anti-4-1BB antibodies has been reported in two lupus models, presumably by suppressing CD4 T cell responses^{20,21}. However, the suppression of a CD4 response is not nondiscriminatory enhanced CD4 T cell response to cancer cells was observed (data not shown). Alternatively, the present disclosure demonstrates that a combination of the two antibodies substantially increased the activity of regulatory T cells. Given the potent effect of Treg in autoimmune diseases, it is quite possible that the uncoupling of autoimmunity and cancer immunity is based on the fact that autoimmune responses are more susceptible to immune regulation.

Anti-CTLA-4 antibodies have been shown to enhance autoimmune diseases in several animal models¹⁵⁻¹⁸. More recently, it has also been reported to induce strong autoimmune disease in cancer patients¹⁹. The present disclosure shows that in cancer-bearing mice, this antibody increased the production of anti-DNA antibodies and deposition of immune complex in the kidney. The disclosure also shows that this antibody can enhance inflammation in the lung. Both side effects are controlled by co-injection of anti-4-1BB antibody. It is of interest to note that while 4-1BB antibody suppressed autoimmune diseases in at least two models^{20,21}, immunotherapy with anti-4-1BB antibody was not totally devoid of autoimmune side effects. In fact, anti-4-1BB antibody actually increased inflammation in the liver. Surprisingly, such inflammation can be suppressed by co-injection with anti-CTLA-4 antibody. These data suggest that side-effects to different organs can be differentially modulated. Although the mechanism of the mutual antagonism of the two antibodies is unclear, such an antagonism in autoimmunity and synergistic effect in cancer rejection suggests that the combination may be of general significance for cancer therapy.

### DOCUMENTS CITED HEREINABOVE

1. Boon, T. et al. Identification of tumour rejection antigens recognized by T lymphocytes. Cancer Surv 13, 23-37 (1992).

2. Boon, T., Cerottini, J. C., Van den Eynde, B., van der Bruggen, P. & Van Pel, A. Tumor antigens recognized by T lymphocytes. Annu Rev Immunol 12, 337-65 (1994).

3. Houghton, A. N. Cancer antigens: immune recognition of self and altered self [comment]. J Exp Med 180, 1-4 (1994).

4. Nanda, N. K. & Sercarz, E. E. Induction of anti-self-immunity to cure cancer. Cell 82, 13-7 (1995).

5. Overwijk, W. W. et al. Tumor regression and autoimmunity after reversal of a functionally tolerant state of self-reactive CD8+ T cells. J Exp Med 198, 569-80 (2003).

6. Albert, M. L. et al. Tumor-specific killer cells in paraneoplastic cerebellar degeneration. Nat Med 4, 1321-4 (1998).

7. Gilboa, E. The risk of autoimmunity associated with tumor immunotherapy. Nat Immunol 2, 789-92 (2001).

8. Ramirez-Montagut, T., Turk, M. J., Wolchok, J. D., Guevara-Patino, J. A. & Houghton, A. N. Immunity to melanoma: unraveling the relation of tumor immunity and autoimmunity. Oncogene 22, 3180-7 (2003).

9. Trcka, J. et al. Redundant and alternative roles for activating Fc receptors and complement in an antibody-dependent model of autoimmune vitiligo. Immunity 16, 861-8 (2002).

10. Leach, D. R., Krummel, M. F. & Allison, J. P. Enhancement of antitumor immunity by CTLA-4 blockade [see comments]. Science 271, 1734-6 (1996).

11. Melero, I. et al. Monoclonal antibodies against the 4-1BB T-cell activation molecule eradicate established tumors. Nat Med 3, 682-5 (1997).

12. May, K. F., Jr., Chen, L., Zheng, P. & Liu, Y. Anti-4-1BB monoclonal antibody enhances rejection of large tumor burden by promoting survival but not clonal expansion of tumor-specific CD8+ T cells. Cancer Res 62, 3459-65 (2002).

13. van Elsas, A., Hurwitz, A. A. & Allison, J. P. Combination immunotherapy of B16 melanoma using anti-cytotoxic T lymphocyte-associated antigen 4 (CTLA-4) and granulocyte/macrophage colony-stimulating factor (GM-CSF)-producing vaccines induces rejection of subcutaneous and metastatic tumors accompanied by autoimmune depigmentation. J Exp Med 190, 355-66 (1999).

14. van Elsas, A. et al. Elucidating the autoimmune and antitumor effector mechanisms of a treatment based on cytotoxic T lymphocyte antigen-4 blockade in combination with a B16 melanoma vaccine: comparison of prophylaxis and therapy. J Exp Med 194, 481-9. (2001).

15. Karandikar, N. J., Vanderlugt, C. L., Walunas, T. L., Miller, S. D. & Bluestone, J. A. CTLA-4: a negative regulator of autoimmune disease. J Exp Med 184, 783-8 (1996).

16. Luhder, F., Chambers, C., Allison, J.P., Benoist, C. & Mathis, D. Pinpointing when T cell costimulatory receptor CTLA-4 must be engaged to dampen diabetogenic T cells. Proc Natl Acad Sci USA 97, 12204-9 (2000).

17. Hurwitz, A. A., Sullivan, T. J., Sobel, R. A. & Allison, J. P. Cytotoxic T lymphocyte antigen-4 (CTLA-4) limits the expansion of encephalitogenic T cells in experimental autoimmune encephalomyelitis (EAE)-resistant BALB/c mice. Proc Natl Acad Sci USA 99, 3013-7 (2002).

18. Piganelli, J. D., Poulin, M., Martin, T., Allison, J. P. & Haskins, K. Cytotoxic T lymphocyte antigen 4 (CD152) regulates self-reactive T cells in BALB/c but not in the autoimmune NOD mouse. J Autoimmun 14, 123-31 (2000).

19. Phan, G. Q. et al. Cancer regression and autoimmunity induced by cytotoxic T lymphocyte-associated antigen-4 blockade in patients with metastatic melanoma. Proc Natl Acad Sci U.S.A. 100, 8372-8377 (2003).

20. Sun, Y. et al. Costirmulatory molecule-targeted antibody therapy of a spontaneous autoimmune disease. Nat Med 8, 1405-13 (2002).

21. Foell, J. et al. CD137 costimulatory T cell receptor engagement reverses acute disease in lupus-prone NZB×NZW F1 mice. J Clin Invest 111, 1505-18 (2003).

22. McHugh, R. S. et al. CD4(+)CD25(+) immunoregulatory T cells: gene expression analysis reveals a functional role for the glucocorticoid-induced TNF receptor. Immunity 16, 311-23 (2002).

23. Takahashi, T. et al. Immunologic self-tolerance maintained by CD25(+)CD4(+) regulatory T cells constitutively expressing cytotoxic T lymphocyte-associated antigen 4. J Exp Med 192, 303-10 (2000).

24. Zheng, B. J. et al. Selective functional deficit in dendritic cell—T cell interaction is a crucial mechanism in chronic hepatitis B virus infection. J Viral Hepat 11, 217-24 (2004).

25. Zhang, Y. & Allison, J. P. Interaction of CTLA-4 with AP50, a clathrin-coated pit adaptor protein. Proc Natl Acad Sci USA 94, 9273-8 (1997).

26. Zheng, G., Wang, B. & Chen, A. The 4-1BB Costimulation Augments the Proliferation of CD4+CD25+ Regulatory T Cells. J Immunol 173, 2428-34 (2004).

27. Hara, I., Takechi, Y. & Houghton, A. N. Implicating a role for immune recognition of self in tumor rejection: passive immunization against the brown locus protein. J Exp Med 182, 1609-14 (1995).

28. Wolchok, J. D. et al. Alternative roles for interferon-gamma in the immune response to DNA vaccines encoding related melanosomal antigens. Cancer Immun 1, 9 (2001).

29. Bowne, W. B. et al. Coupling and uncoupling of tumor immunity and autoimmunity. J Exp Med 190, 1717-22 (1999).

30. Wilcox, R. A. et al. Provision of antigen and CD137 signaling breaks immunological ignorance, promoting regression of poorly immunogenic tumors. J Clin Invest 109, 651-9 (2002).

31. Walunas, T. L. et al. CTLA-4 can function as a negative regulator of T cell activation. Immunity 1, 405-13 (1994).

Testing of Anti-Human CTLA4

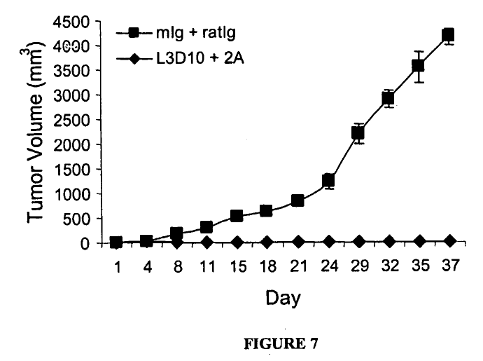

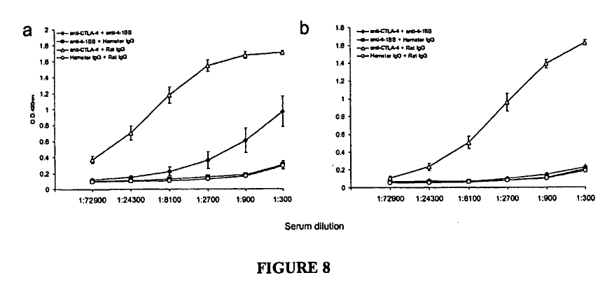

We have recently produced a human CTLA4 gene knock-in mouse in which the mouse CTLA4 gene is replaced with its human counterpart (1). This mouse allowed us to test if the anti-tumor effect of the anti-human CTLA4 antibodies can be enhanced by anti-4-1BB antibody. As shown in FIG. 8, while both anti-human CTLA4 (L3D10) and anti-4-1BB antibody (2A) alone caused delayed tumor growth, combination of the two antibodies resulted in most significant tumor rejection. In fact, 5/7 mice in the group treated with the two antibodies never developed tumors, while all mice in other 3 groups developed tumors. To test whether the double antibody treated mice were immune to further tumor cell challenge, we challenged them with tumor cells at 110 days after their first tumor cell challenge. As shown in FIG. 7, all of the five double antibody-treated mice that have rejected the tumor cells in the first round remained tumor-free, while control naive mice have progressive tumor growth. Taken together, our data demonstrated superior effect of combination therapy will likely be applicable to anti-human CTLA-4 antibody.

One of the obstacles to repeated antibody therapy is the enhancement of host antibody responses to the therapeutic antibodies (2, 3). Since 4-1BB was known to reduce antibody response to proteins, we evaluated the effect of anti-4-1BB antibodies on host response to anti-CTLA4 antibodies. As shown in FIG. 8, very little, if any anti-antibody response was detected in mice treated with either control IgG or 4-1BB. Consistent with the ability of anti-CTLA-4 mAb to facilitate CD4 T cell responses (4), mice treated with anti-CTLA-4 plus rat IgG developed strong host antibody responses against the administered 4F10 and rat IgG (FIGS. 8a & 8b). This response was reduced by more than 30-fold when anti-4-1BB was co-administered with anti-CTLA4 mAb. These data demonstrate another important feature of combination therapy, namely reduction of host immune response to the therapeutic agent.

### DOCUMENTS CITED IN TESTING OF ANTI-HUMAN CTLA4

1. Lute K D, May K F, Lu P, et al. Human CTLA-4-knock-in mice unravel the quantitative link between tumor immunity and autoimmunity induced by anti-CTLA-4 antibodies. Blood 2005 (In Press).

2. Schroff, R. W., Foon, K. A., Beatty, S. M., Oldham, R. K., and Morgan, A. C., Jr. 1985. Human anti-murine immunoglobulin responses in patients receiving monoclonal antibody therapy. Cancer Res 45:879-885.

3. Sharkey, R. M., Juweid, M., Shevitz, J., Behr, T., Dunn, R., Swayne, L. C., Wong, G. Y., Blumenthal, R. D., Griffiths, G. L., Siegel, J. A., et al. 1995. Evaluation of a complementarity-determining region-grafted (humanized) anti-carcinoembryonic antigen monoclonal antibody in preclinical and clinical studies. Cancer Res 55:5935s-5945s.

4. Kearney, E. R., Walunas, T. L., Karr, R. W., Morton, P. A., Loh, D. Y., Bluestone, J. A., and Jenkins, M. K. 1995. Antigen-dependent clonal expansion of a trace population of antigen-specific CD4+ T cells in vivo is dependent on CD28 costimulation and inhibited by CTLA-4. J Immunol 155:1032-1036.

Other embodiments of the invention will be apparent to those skilled in the art from consideration of the specification and practice of the invention disclosed herein. It is intended that the specification and examples be considered as exemplary only, with a true scope and spirit of the invention being indicated by the claims.

What is claimed is:

1. A method of treating cancer comprising administering to a patient in need anti-4-1BB antibody and anti-CTLA4 antibody in amounts effective to produce an anti-cancer effect, wherein the administration of anti-4-1BB antibody and anti-CTLA4 antibody result in a lower level of autoimmunity as compared to administration of either anti-CTLA4 or anti-4-1BB antibody alone.

2. A method for reducing an autoimmune side effect in administration of anti-CTLA4 antibodies comprising administering an effective amount of anti-4-1BB antibody to a patient anticipating or experiencing anti-CTLA4 autoimmune side effects.

3. A method for reducing an autoimmune side effect in administration of anti-CTLA4 antibodies comprising administering an effective amount of anti-CTLA4 antibody to a patient anticipating or experiencing anti-4-1BB autoimmune side effects.

4. A method for enhancing cancer immunity in a patient while reducing autoimmunity in said patient comprising administering anti-4-1BB antibody and anti-CTLA4 antibody in effective amounts to a patient in need of treatment.

5. The method according to any of claims 1 to 4, wherein the anti-cancer effect is an effect chosen from decreased tumor burden, decreased metastasis, decreased tumor growth, and reduction in new tumor formation.

6. The method according to any of claims 1 to 4, wherein autoimmunity is measured by testing for anti-DNA antibodies or inflammation of noncancerous tissues.

7. The method according to any of claims 1 to 4, wherein the anti-4-1BB antibody and anti-CTLA4 antibody are administered simultaneously.

8. The method according to any of claims 1 to 4, wherein the anti-4-1BB antibody and anti-CTLA4 antibody are administered at different times.

9. A composition for treating cancer comprising effective amounts of anti-4-1BB antibody and anti-CTLA4 antibody, and at least one pharmaceutically acceptable excipient, wherein the amount of the antibodies in the composition is sufficient to produce a lower level of autoimmunity as compared to a composition of either antibody alone.

10. A method of reducing host immune response against a therapeutic antibody by administering to the host an anti-4-1BB antibody.

11. The method according to claim 10, where the therapeutic antibody is targeted at a CTLA4 molecule.
